(19) United States
(12) Patent Application Publication
(10) Pub. No.: US 2023/0363376 A1
(43) Pub. Date: Nov. 16, 2023

(54) ORGAN PERFUSION PUMP RESERVOIR FILTER DEVICE

(71) Applicant: Burnett Stephens KELLY, Sacramento, CA (US)

(72) Inventor: Burnett Stephens KELLY, Sacramento, CA (US)

(21) Appl. No.: 18/311,418

(22) Filed: May 3, 2023

Related U.S. Application Data
(63) Continuation-in-part of application No. 16/482,402, filed on Jul. 31, 2019, now abandoned.
(60) Provisional application No. 62/454,945, filed on Feb. 6, 2017.

(51) Int. Cl. A01N 1/02

(52) U.S. Cl. CPC A01N 1/0247; A01N 1/0278; A61B 2017/00969

(57) ABSTRACT

An apparatus for cleaning transplant organs comprising an organ perfusion circuit and perfusion solution filtration circuit that removes virus and bacteria. The perfusion pump circuit has a cassette for containing perfusion fluid and an organ and a perfusion pump to circulate perfusion fluid in and out of the cassette. A perfusion solution filtering device has a filter and a pump coupled to the cassette to pump perfusion fluid out of the cassette, through the filter, and back into the cassette.

### PRIOR RELATED APPLICATIONS

This application is a continuation-in-part of application Ser. No. 16/482,402 (published as US2019357527), filed Jul. 31, 2019, which is a National Stage entry of PCT/US18/13785, filed Jan. 16, 2018, which claims priority to U.S. provisional Application 62/454,945, filed Feb. 6, 2017. Each is incorporated by reference in its entirety for all purposes.

### FEDERALLY SPONSORED RESEARCH STATEMENT

Not applicable.

### FIELD OF THE DISCLOSURE

This invention provides devices and methods of the cleaning of infected organs for use in transplants, thus, increasing the pool of available transplant organs.

### BACKGROUND OF THE DISCLOSURE

Transmission of viral and bacterial disease from organ donors to recipients is a significant transplant rate-limiting problem. As one example, hepatitis C virus (HCV) is a 30-65 nanometer viral particle that infects millions of people in the US and is found in approximately 15-20% of potential organ donors, plus transmission of HCV is thought to occur with minimal viral inoculum. Thus, to prevent transmission of virus from an infected organ donor to an uninfected waitlisted recipient, many otherwise functional organs (particularly kidneys) are discarded due to virus being detected in the donor. Other donor organs such as the lungs and heart are rarely even considered for transplant due to the significant morbidity that can be incurred in recipients of these organs. Although antiviral and antibacterial medical therapies exist to treat such infections, strategies to minimize the inoculum could significantly increase the number of available life-saving organs. Thus, a tremendous opportunity to utilize viable organs from this underappreciated patient source population is not being optimally utilized.

What is needed in the art are methods and devices for preparing organs for transplant that remove bacteria and viruses and allow the use of otherwise discarded transplantable organs. This invention provides such a viable method and devices for same.

### SUMMARY OF THE DISCLOSURE

HCV, Hepatitis B, and HIV represent the most common lethal transmittable blood-borne viruses, while several antibiotic resistant organisms can also limit utilization of organs from infected donors. HCV affects all organ types, and for the purposes of simplifying this application is used as an example of the potential benefit of the proposed invention. Viable and transplantable organs from HCV⁺ organ donors have a significantly higher discard rate than non-HCV organ donors because of the theoretical risk of HCV transmission to a viral load negative or different HCV genotyped recipient.

In many U.S. organ procurement organizations (OPOs), HCV⁺ donors may represent 5-40% of organ donors during a year. There are presently over 120,000 waitlisted patients with an estimated death rate of 7-8 patients dying daily without the opportunity for transplant. A majority of these waitlisted patients are awaiting renal transplant. For hemodialysis patients, there is an estimated risk of annual mortality exceeding 50%. The life benefit of renal transplantation has been realized within 3 years of wait-listing, and further life-year benefit has been demonstrated for certain end stage renal disease (ESRD) patients who receive pre-emptive transplant prior to initiating dialysis. With an estimated 15-20% of the US renal transplant waitlist being composed of HCV⁺ patients, there is some theoretical benefit to identifying a strategy to safely utilize any quality kidney (including from an HCV⁺ donor).

It is further estimated that hemodialysis patients have a 40-50% incidence of HCV. In addition, an increasing percentage of organ donors engage in high-risk behaviors such as IV drug use and sexual/hygiene practices that may predispose them to carrying asymptomatic viral hepatitis that was not previously diagnosed prior to conducting the organ donation evaluation.

During the organ donation evaluation, the testing identifies antibodies (acute IgM and chronic carrier IgG fractions) to viral infections as well as the presence of active nuclear antigens (for example, by nucleic acid testing (NAT)). Typically, no viral load or genotype testing is conducted due to cost and impracticality in the present accepted organ allocation process.

The timing of antiviral treatment of HCV⁺ recipients post-transplant is not yet standardized, and may be outcome limited by mixed donor/recipient HCV genotypes, and immunosuppression strategies over the life-span of the organ.

Pulsatile Perfusion: Cold pulsatile perfusion for kidneys was introduced into clinical practice in the 1990's, with the hypothesis that perfusion of the renal vasculature by either continuous roller pump or intermittent pulsatile flow dynamics would maintain cellular metabolic arrest while “opening” the microvasculature of the kidney. Organ preservation solution is “pumped” through a sterile enclosed fluid circuit composed of the pump, non-distensible tubing, a porous air-trap and large particle filter, and affluent/effluent circuit flow probes.

With continued experience, the flow/pressure dynamics have been interpreted to provide additional predictive information on the post-reperfusion function of the kidney in the recipient. The ideal kidney for transplant would be defined as a kidney with a Kidney Donor Predictive Index (KDPI) score <80, a favorable renal biopsy result, normal renal anatomy, low pump resistance (<0.2), and renal artery flow >100.

Herein we provide a device and method for cleaning a kidney and other organ for transplant use by adding a cleaning circuit to the perfusion circuit. The cleaning circuit has one or more filters that remove large particles, such as bacteria and virus, and returns cleaned perfusion fluid to the perfusion circuit. Clean perfusion fluid returns to the organ, passing therethrough, and gradually drawing out more virus and bacteria, which is then circulated again to the filter circuit. The continued flow of the two circuits gradually removes most, if not all virus and bacteria from the organ, so that it can be safely transplanted.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as labels, instructions for use, power couplings, on/off buttons, indicator lights, temperature controls, and the like.

The following abbreviations are used herein:

| ABBREVIATION | TERM |
|---|---|
| ESRD | End Stage renal disease |
| HCV | Hepatitis C virus |
| KDPI | Kidney Donor Predictive Index |
| OPO | Organ procurement organization |
| NAT | Nucleic acid amplification testing |
| SRV | Sustained viral response |

### BRIEF DESCRIPTION OF THE DRAWINGS

FIG. 1A. is a block diagram of perfusion pump circuit and solution viral clearance pump.

FIG. 1B. is a variant of FIG. 1A where the cleaned fluid directly enters the perfusion circuit line.

FIG. 2 is a cross section of the filter arrangement. Only one filter is shown, but of course, multiple filters could be used, preferably in decreasing pore size along the direction of flow.

FIG. 3 shows a perspective view of the layout of FIG. 1A.

### DETAILED DESCRIPTION

The following detailed description illustrates embodiments of the present disclosure. These embodiments are described in sufficient detail to enable a person of ordinary skill in the art to practice these embodiments without undue experimentation. It should be understood, however, that the embodiments and examples described herein are given by way of illustration only, and not by way of limitation. Various substitutions, modifications, additions, and rearrangements may be made that remain potential applications of the disclosed techniques. Therefore, the description that follows is not to be taken as limiting on the scope of the appended claims. In particular, an element associated with a particular embodiment should not be limited to association with that particular embodiment but should be assumed to be capable of association with any embodiment discussed herein.

The premise of the preservation pump reservoir filtering system is to filter deleterious viral (and potentially bacterial) particles from the organ effluent during the pulsatile preservation process in an attempt to lower the remaining inoculum in the organ below the threshold of clinical disease transmission. By diminishing the circulating viral and bacterial load in the organ perfusion circuit and replenishing fresh preservation solution, the transplanted viral/microbial inoculum will be diminished, rendering the recipient more likely to be immune to low-dose viral/microbial exposure, and more likely to be responsive to post-transplant antiviral/antibiotic therapy.

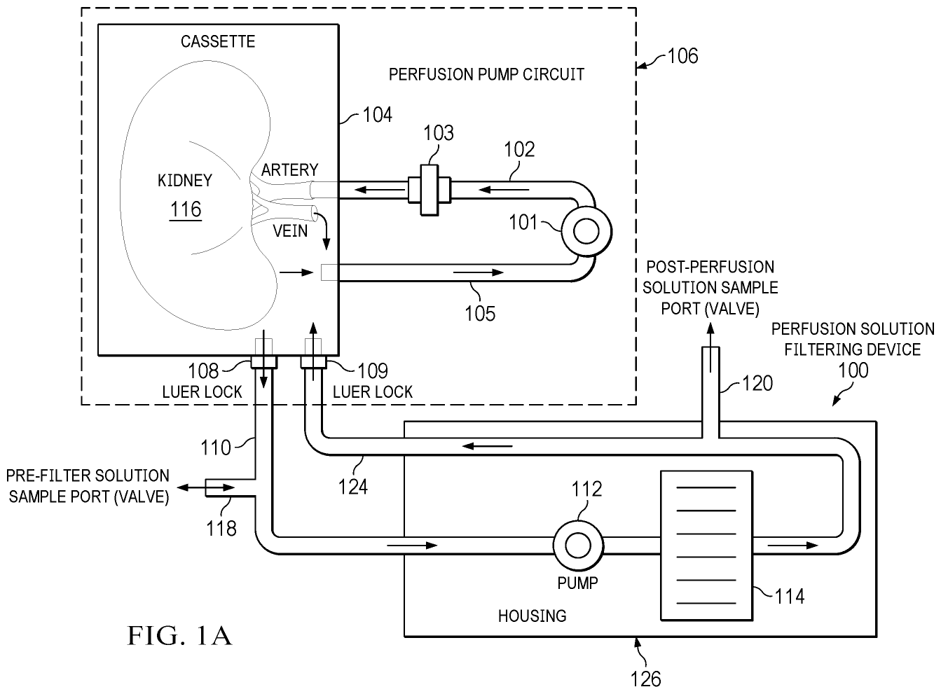

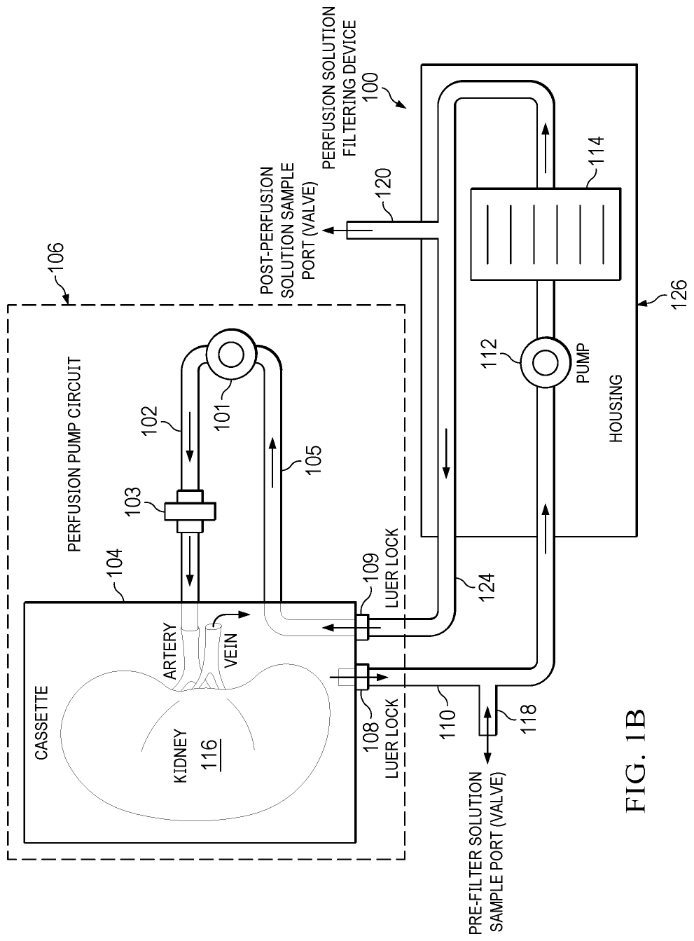

A device for this purpose is shown in a top down view of FIG. 1A wherein the perfusion solution filtering device 100 is typically an external device that connects to the sterile organ cassette 104 of the perfusion pump circuit 106 via a Luer lock port 108/109, or similar device, herein shown connecting on the organ cassette 104 wall. Alternatively, the sterile organ cassette 104 could be accessed through a different entry point, such as the lid.

Ideally, the perfusion pump is a peristaltic (pulsating) pump, such as the micro-perfusion pump by Thomas Scientific® or the FCS micro-perfusion pump by Bioptechs®. Unlike most peristaltic pumps that are driven by stepper motors, the micro-perfusion pump by Thomas Scientific® is driven by a tachometer regulated, multi-stage DC gear motor. This assures a smooth analog rotation of the roller spindle, free of instantaneous steps. Other similar pumps include the Quantum Roller Pump by Spectrum Medical®, the Organ Recovery Systems (ORS) and more recently ex-situ normothermic perfusion pumps.

Preferred first and second pumps roll over the tubing containing the perfusion fluid and thus never contact the fluid, eliminating the need to address pump sterility. The ideal pump can operate in push or pull mode, is small and does not require flow rate calibration.

The cassette can be stored on ice, or the one or more units fitted with temperature control units to keep the organ cool during perfusion. In addition, some perfusion circuits have an in-line oxygenator feature.

In an alternate embodiment (not shown), the perfusion pump circuit 106 and filter 114 could be combined into a single unit. In such a case, the filter may be installed in the perfusion fluid return line 105 before perfusion pump 101. It could also be placed on fluid input line 102. However, in FIG. 1A and FIG. 1B the filter circuit is separate from the perfusion circuit, allowing the addition of this unit to already existing perfusion pump circuits for any organ, and obviating the purchase of an entire new unit. This design also allows continuous and even flow through the filter 114, rather than the peristaltic flow of the perfusion pump 101, and is thus preferred.

Sterile silastic tubing 110 inserts through the wall port or other entry point, preferably down into the most gravity dependent portion of the organ reservoir 104 (e.g., the ports are low in the housing). Tubing 110 sterilely connects to the perfusion solution filtering device 100 containing a pump 112 that will draw fluid from the organ cassette 104 and push it through a filter 114 (herein tested an Aethlon Medical Filter). In FIG. 1 we show the pump 112 before the filter 114, but its placement can be before or after.

Preferred tubing for both units is silastic tubing—a silicon tubing. Ideally the tubing will be platinum-cured, nonpyrogenic, bacteria and fungus-resistant, extremely low extractables, and does not impart a taste or odor, such as that provided by VWR® or Bioseal®

The filtered effluent will then return to the sterile organ cassette 104 via return tubing 124 for continued pulsatile preservation of the organ 116, herein shown a kidney. At the inflow 118 and outflow 120 channels of the device 102 will be Luer lock specimen sampling ports, or similar devices, allowing the user to infuse therapy or fresh preservation solution to improve particle clearance or to sample the perfusion fluid and measure viral or bacterial load.

The perfusion solution filtering device 100 includes input tubing 110 and the output tubing 124, filter 114 and pump 112. Sample ports 118, 120 can be primed with 250-500 cc of fresh perfusion solution to promote fluid exchange without introduction of air into the system.

Once the organ perfusion and filtration is completed, the filtering pump lines 110, 124 can be removed, and the ports capped for removal of the organ from the cassette for transplantation. The filter cartridge 114 and tubing 102, 110, 124 are a disposable unit that can be removed from the filter pump housing 126 for hazardous waste discard at the completion of use. The pump 112 ideally does not require any calibration or additional maintenance. In addition, the housing is not strictly essential, but is included to contain and protect filter 114 and lines 110/124 in a neat manner.

The perfusion pump perfusion cassette is a single contained entity that contains in-line sensors that measure pressure, flow, and resistance. Thus, the filter pump does not need any inline sensors, and therefore does not require any additional calibration to achieve accuracy of these measurements.

FIG. 1B is very similar to 1A, but the return line 124 is directly connected to return line 105.

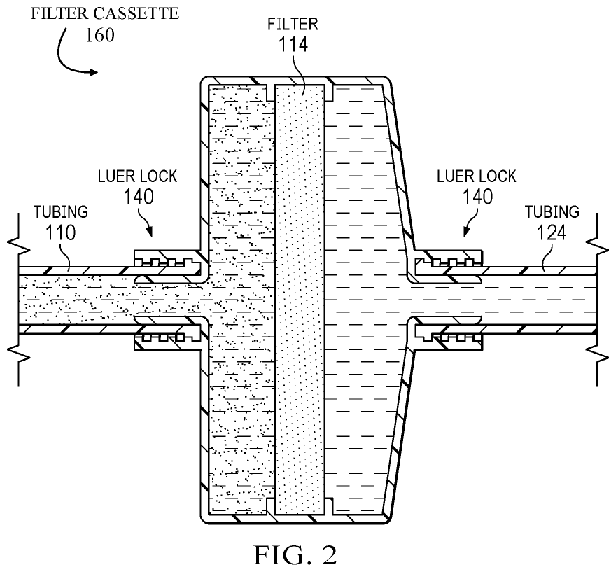

FIG. 2 shows a cross section of the filter 114 in filter cassette 160, and connectors 140 to tubing 110/124. Filter 114 is chosen to filter out viruses, and thus pore size is chosen to be small enough to catch the virus of concern. A virus may be as small as 20 nm (e.g., Parvovirus), thus, the pore size may be <20 nm or e.g., 15 nm. However, smaller pores restrict flow so it may be preferred to provide a variety of filters in different pore sizes to capture different viruses. Thus, an HCV specific filter would have pores <30-65 nm, e.g., 25 nm. It may also be desired to use an array of filters or decreasing pore size (e.g., 100 nm>60 nm>25 nm) so as to reduce the need to replace filters.

Generally, any filter with pores small enough to catch HCV will also filter out blood cells and bacteria, such as M. tuberculosis, E. coli, and S. Aureus. However, the organ is being maintained with perfusion fluid, and the blood cells need not be retained.

Suitable filters may include highly asymmetric Viresolve® Pro and Viresolve® NFP filters from MilliporeSigma®, or the relatively homogeneous Ultipor DV20 and Pegasus™ SV4 virus filters from Pall®.

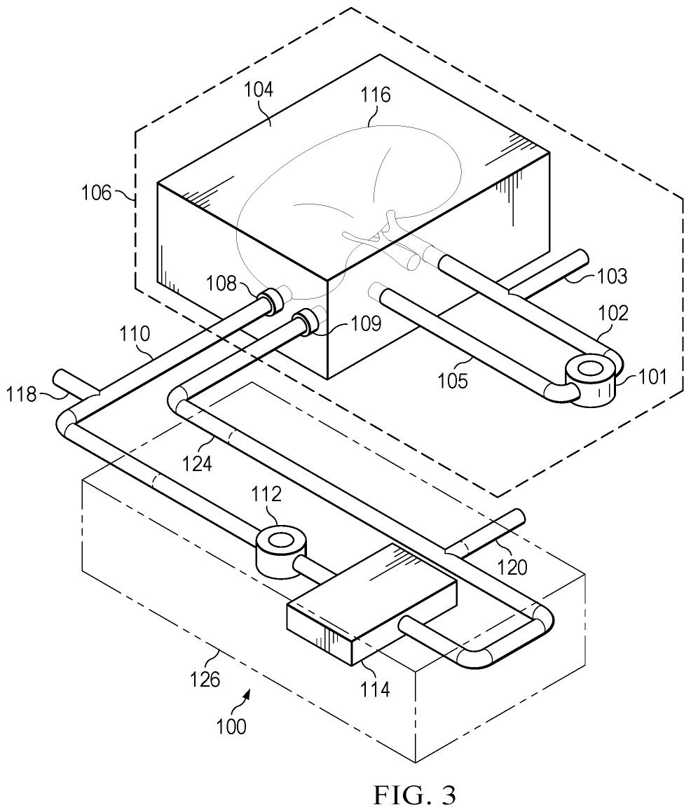

FIG. 3 shows the system of FIG. 1A in perspective. All the same numerals are employed.

While the description and FIG. 1 herein illustrates the use of the inventive apparatus with a kidney, it will be understood that the system can be used with all potential perfused organs. Further, the system is designed to filter all transmissible virus and bacteria and is not limited to those specific virus and bacteria mentioned herein.

In one aspect, an apparatus includes a perfusion pump circuit having a cassette for containing perfusion fluid and a perfusion pump to circulate perfusion fluid in and out of the cassette and organ. The apparatus includes a perfusion solution filtering device having a filter or hemofilter and a pump coupled to the cassette to pump perfusion fluid out of the cassette, through the filtration unit, and back into the cassette.

The apparatus may include input tubing connecting the perfusion pump circuit to the perfusion solution filtering device by which fluid is drawn from the perfusion pump circuit and delivered to the pump and output tubing connecting the perfusion solution filtering device to the perfusion pump circuit by which fluid is pumped from the filter to the perfusion pump circuit.

The apparatus may include a port into the input tubing, and a port into the output tubing. The perfusion solution filtering device, the input tubing, and the output tubing may be disposable. The input tubing may be inserted into the most gravity dependent (lowest) portion of the perfusion pump circuit. The pump in the perfusion solution filtering device may not require calibration.

In one aspect, a method includes coupling a perfusion pump circuit to a perfusion solution filtering device, each of these as describe herein. The perfusion pump circuit may have a cassette for containing perfusion fluid and an organ to be perfused and a perfusion pump to circulate perfusion fluid in and out of the cassette. The perfusion solution filtering device may have a hemofilter or any filter configured to remove virus, and bacteria and a pump coupled to the cassette to pump perfusion fluid out of the cassette, through the filter, and back into the cassette.

Implementations may include one or more of the following.

The method may include running the perfusion solution filtering device until the organ perfusion is completed and disconnecting and discarding the perfusion solution filtering device. Coupling the perfusion pump circuit to the perfusion solution filtering device may include coupling the perfusion pump circuit to the perfusion solution filtering device with an input tubing by which fluid is drawn from the perfusion pump circuit and delivered to the pump and coupling the perfusion solution filtering device to the perfusion pump circuit with an output tubing by which fluid is pumped from the hemofilter to the perfusion pump circuit. The method may include running the perfusion solution filtering device until the organ perfusion is completed and disconnecting and discarding the input and output tubing and filter.

The word “coupled” herein means a direct connection or an indirect connection.

The following are each incorporated by reference in its entirety for all purposes.

US2014017666 Filtration in organ perfusion apparatus.

US2010092939 (U.S. Pat. No. 9,113,624) System and method for perfusing biological organs.

US2006129082 Selective plasma exchange therapy.

US20200080050 US2021139843 Systems and methods for growing cells in vitro.

U.S. Pat. Nos. 5,549,674, 5,686,289 Methods and compositions of a bioartificial kidney suitable for use in vivo or ex vivo.

1. An organ preservation and virus filtration apparatus, said apparatus consisting essentially of:
a) a perfusion pump circuit having:
i) a cassette configured to contain an organ and a perfusion fluid for preservation of said organ; and
ii) a first pump and a first tubing fluidly coupled to said pump and to said cassette to circulate said perfusion fluid through said organ and back to said first pump;
b) a perfusion solution filtering device having:
i) a filter configured to remove virus and bacteria from said perfusion fluid; and
ii) a second pump and a second tubing fluidly coupled to said cassette and to said second pump to circulate said perfusion fluid out of said cassette, through said filter to remove virus and bacteria and send cleaned perfusion fluid back into said cassette.

2. The apparatus of claim 1, wherein said first pump is a peristaltic pump.

3. The apparatus of claim 1, further comprising a sampling port on said second tubing.

4. The apparatus of claim 1, wherein said second tubing comprises:
a) input tubing connecting said perfusion pump circuit to said perfusion solution filtering device; and
b) output tubing connecting said perfusion solution filtering device to said perfusion pump circuit.

5. The apparatus of claim 4, further comprising:
a) a sampling port in said input tubing, and
b) a sampling port in said output tubing.

6. The apparatus of claim 1, said perfusion solution filtering device further comprising a housing to contain said filter, said pump and a portion of said second tubing.

7. An organ preservation and filtration apparatus, said apparatus consisting essentially of:
a) a perfusion pump circuit having:
i) a cassette configured to contain an organ and a perfusion fluid for preservation of said organ; and
ii) a first pump and a first tubing to circulate said perfusion fluid into and out of said cassette and said organ;
b) a perfusion solution filtering device having:
i) a filter configured to remove virus from said perfusion fluid; and
ii) a second pump and a second tubing coupled to said cassette and said second pump to circulate said perfusion fluid out of said cassette, through said filter to remove virus, and circulate filtered perfusion fluid without said virus back into said cassette, thereby cleaning said organ for use in transplant surgery.

8. The apparatus of claim 7, said second tubing comprising:
a) input tubing fluidly coupling said perfusion pump circuit to said perfusion solution filtering device;
b) output tubing fluidly coupling said perfusion solution filtering device to said perfusion pump circuit;
c) a sample port in said input tubing, and
d) a sample port into said output tubing.

9. The apparatus of claim 7, said perfusion solution filtering device further comprising a housing to contain said filter, said pump and a portion of said second tubing.

10. An organ preservation and virus removal method, said method comprising:
a) coupling a perfusion pump circuit to a perfusion solution filtering device;
i) wherein the perfusion pump circuit has:
(1) a cassette for containing an organ and a perfusion fluid to preserve said organ, and
(2) a perfusion pump and a first tubing to circulate said perfusion fluid into said organ and said cassette and out of said cassette;
ii) wherein the perfusion solution filtering device has:
(1) a filter configured to remove virus and bacteria from said perfusion fluid; and
(2) a pump and a second tubing to circulate perfusion fluid out of the cassette, through said filter to remove virus, and circulate filtered perfusion solution back into said cassette;
b) placing an organ comprising a virus plus perfusion fluid into said cassette;
c) operating said perfusion pump circuit to perfuse said organ;
d) operating said perfusion solution filtering device to filter said perfusion fluid, thereby removing virus from said organ to produce a cleaned organ; and
e) removing said cleaned organ from said cassette.

11. The method of claim 10, wherein said organ is a kidney.

12. The method of claim 10, wherein said virus is a hepatitis virus.

13. The method of claim 10, wherein said organ is a kidney and said virus is a hepatitis virus.

14. An organ transplant method, said method comprising:
a) testing a donated organ for virus to identify a virus positive organ;
b) filling said cassette of said apparatus of claim 1 with perfusion fluid;
c) placing said virus positive organ into said cassette and connecting said first tubing to an artery of said organ;
d) operating said perfusion pump circuit to perfuse said organ with said perfusion fluid;
e) operating said perfusion solution filtering device to filter said perfusion fluid, thereby removing virus from said organ and providing a cleaned organ;
f) removing said cleaned organ from said cassette; and
g) transplanting said cleaned organ into a patient needing an organ transplant.

15. The method of claim 14, wherein said organ is a kidney.

16. The method of claim 14, wherein said virus is a hepatitis virus.

17. The method of claim 14, wherein said organ is a kidney and said virus is a hepatitis virus.

18. An organ transplant method, said method comprising:
a) testing a donated organ for virus to identify a virus positive organ;
b) filling said cassette of said apparatus of claim 7 with perfusion fluid;
c) placing said virus positive organ into said cassette and connecting said first tubing to an artery of said organ;
d) operating said perfusion pump circuit to perfuse said organ with said perfusion fluid;
e) operating said perfusion solution filtering device to filter said perfusion fluid, thereby removing virus from said organ and providing a cleaned organ;
f) removing said cleaned organ from said cassette; and
g) transplanting said cleaned organ into a patient needing an organ transplant.

19. The method of claim 18, wherein said organ is a kidney.

20. The method of claim 18, wherein said organ is a kidney and said virus is a hepatitis virus.
